(19) United States
(12) Patent Application Publication
(10) Pub. No.: US 2004/0033483 A1
(43) Pub. Date: Feb. 19, 2004

(54) Cell diagnosing method, and device and apparatus use for it

(76) Inventors: Hiroaki Oka, Osaka (JP); Ryuta Ogawa, Osaka (JP)

Correspondence Address: SNELL & WILMER, ONE ARIZONA CENTER, 400 EAST VAN BUREN, PHOENIX, AZ, 850040001

(21) Appl. No.: 10/398,828

(22) PCT Filed: Aug. 5, 2002
(86) PCT No.: PCT/JP02/07984

(30) Foreign Application Priority Data
Aug. 9, 2001 (JP) 2001-242861

(51) Int. Cl. C12Q 1/00

(52) U.S. Cl. 435/4

(57) ABSTRACT

A method for diagnosing a state and a characteristic of a cell comprising the steps of providing a reaction system for measuring a physicochemical characteristic of the cell, placing a sample including a cell membrane fraction of the cell in the reaction system, applying a stimulus to the sample, obtaining an index of the physicochemical characteristic of the sample, and diagnosing the state of the cell with reference to the index. The placement environment or characteristic of the reference electrode is different from the placement environment or characteristic of the measuring electrode. The substrate comprises a through hole for placing the sample thereon, and the sample is placed between the reference electrode and the measuring electrode and in the vicinity of the through hole.

### TECHNICAL FIELD

The present invention relates to a cell diagnosis method for determining the state of a subject cell, and a device and an apparatus for use in the same.

### BACKGROUND ART

Conventionally, diagnosis of pathologies using biopsy samples or body fluid has been carried out using staining methods with various antibodies.

Unfortunately these methods may produce different diagnosis results according to the experience of the pathologist, or diagnostic errors may occur depending on antibody titer, and the methods are very time consuming.

### DISCLOSURE OF THE INVENTION

The present invention is provided to solve the above-described problems, and provides a cell diagnosis method cable of processing a large amount of samples easily and quickly without a diagnostic error by measuring changes in physicochemical characteristics of a cell, which are associated with mutation in the cell, and a device and an apparatus for use in the method.

The present invention relates to a method for diagnosing a state and a characteristic of a cell, comprising the steps of providing a reaction system for measuring a physicochemical characteristic of the cell, placing a sample including a cell membrane fraction of the cell in the reaction system, applying a stimulus to the sample, obtaining an index of the physicochemical characteristic of the sample, and diagnosing the state of the cell with reference to the index.

The diagnosis step may comprise comparing the index with an index of the physicochemical characteristic of a control sample.

Representatively, the stimulus may be selected from chemical substances, proteins, amino acids, voltage pulses, current pulses, electromagnetic waves, and laser light.

The invention relates to the device for use in the reaction system, comprising a substrate for placing the sample thereon, a reference electrode, and a measuring electrode. The placement environment or characteristic of the reference electrode is different from the placement environment of characteristic of the measuring electrode.

Representatively, the substrate comprises a through hole for placing the sample thereon; and the sample is placed between the reference electrode and the measuring electrode and in the vicinity of the through hole.

Representatively, the placement environment or characteristic of the reference electrode and the placement environment or characteristic of the measuring electrode are the volumes of regions in which the reference electrode and the measuring electrode are disposed, respectively, and the volume of the region in which the measuring electrode is disposed is smaller than the volume of the region in which the reference electrode is disposed.

Representatively, in the method, the step of providing the reaction system comprises supplying a sufficient amount of an electrolyte to the device that the reference and measuring electrodes are immersed in the electrolyte, and the placing step comprises positioning the sample on a through hole of the device. The amount of the electrolyte immersing the reference electrode is greater than the mount of electrolyte immersing the measuring electrode.

Representatively, the device further comprises a cell culturing section disposed on the substrate, and a cell suctioning section disposed under the substrate. The cell culturing section is formed by a partitioning member and the substrate.

Representatively, the reference electrode is disposed on an inner wall of the partitioning member.

Representatively, the index obtaining step comprises: (a) recording a physicochemical signal emitted by the sample as time-series signal values; (b) sampling the recorded time-series signal values to obtain a plurality of groups of extracted data consisting of a plurality of values, and calculating a standard deviation of each group of data; (c) calculating an average value of the standard deviations; and (d) referring to the average as the index.

Representatively, the index obtaining step comprises: (a) recording a physicochemical signal emitted by the sample as time-series signal values; (b) sampling the recorded time-series signal values to obtain a plurality of groups of extracted data consisting of a plurality of values, and calculating a standard deviation of each group of data; (c) dividing the standard deviations into a plurality of classes having a predetermined size of standard deviation as a unit, and obtaining a distribution indicating the physicochemical characteristic of the sample; (d) approximating the distribution to a normal distribution; and (e) calculating an average and a half width of the resultant normal distribution, and referring to the average of the half width as the indexes.

In the method, the steps (b) to (e) are repeated a plurality of times, the number of the time-series signal values to be sampled is changed in each repetition to obtain a plurality of normal distributions, and the index is selected from averages and half-widths of the normal distributions.

In the method, there may be a plurality of reaction systems for measuring a physicochemical characteristic of the cell, and before the step (b), the method may further comprise adding up the time-series signal values emitted by a plurality of samples placed in the reaction systems.

In the method, before the step (a), the method may further comprise simultaneously stimulating the samples placed in the reaction systems.

The index obtaining step may comprise: (a) recording a physicochemical signal emitted by the sample as time-series signal values; (b) sampling the time-series signal values to obtain a plurality of groups of extracted data consisting of a plurality of values, and calculating a standard deviation of each group of extracted data; (c) dividing the standard deviations into a plurality of classes having a predetermined size of standard deviation as a unit, and obtaining a distribution indicating the physicochemical characteristic of the sample; (d) approximating the distribution by curvilinear approximating analysis selected from the group consisting of exponential decreasing analysis, exponential increasing analysis, Gaussian distribution, Lorentz distribution, o′ analysis, multiple peak analysis, and nonlinear analysis; and (e) obtaining an index of the physicochemical characteristic of the sample based on gradients before and after a peak on the approximated curve obtained by the step (d).

The sample in the step (b) may be carried out a plurality of times from initial data a, which is one of the time-series signal values, in a time-series manner and a plurality of times from data b recorded at a predetermined time after the initial data a in a time-series manner.

The index obtaining step may comprise: (a) recording a physicochemical signal emitted by the sample as time-series signal values; (b) sampling the time-series signal values to obtain a plurality of groups of extracted data consisting of a plurality of values, and calculating a standard deviation of each extracted data; (c) sampling the resultant standard deviations to obtain a plurality of groups of extracted standard deviations consisting of a plurality of values, and calculating an average of each of the plurality of groups of extracted standard deviations; and (d) obtaining an index of the physicochemical characteristic of the sample based on a time of occurrence of the time-series signal value when the average reaches a predetermined threshold.

Representatively, the step (a) may be performed in the presence of a standard chemical substance having a known action of the sample and in the presence of a chemical substance to be tested, and the steps (b) to (e) are repeated by changing the concentrations of the standard chemical substance and the chemical substance to be tested, and the diagnosing step may further comprise comparing an index obtained in the presence of the standard chemical substance with an index obtained in the presence of the chemical substance to be tested.

The present invention also relates to a cell diagnosis apparatus, comprising a reaction system comprising the cell diagnosis device, and means for detecting the physicochemical characteristic of the sample.

The present invention also relates to a cell diagnosis analyzing chip, comprising a substrate for placing a sample including a cell membrane fraction of a cell thereon, a reference electrode, and a measuring electrode. The substrate has a. tissue disruption section, b. cell culturing section, c. sensor section, d. stimulus applying section, e. signal amplifying section, f. signal processing section, g. data displaying section, and h. control panel section, thereby displaying the state of the sample.

The a. tissue disruption section may comprise a microfilter having a diameter of 1 to 100 μm.

The b. cell culturing section may have temperature control means.

The b. cell culturing section may further have the humidity control means.

The temperature control means may have a Peltier device or an IH heater.

### BRIEF DESCRIPTION OF THE DRAWINGS

FIG. 1 is a schematic diagram showing a cell diagnosing device according to an embodiment of the present invention. Reference numerals in FIG. 1 indicate the following members. 1: cell diagnosis device, 2: SOI substrate, 3: well, 4: measuring electrode, 5: culture solution, 6: depression, 7: through hole, and 8: reference electrode.

FIG. 2 is a schematic diagram showing a cell diagnosis device according to another embodiment of the present invention. Reference numerals in FIG. 2 indicate the following members. 10: cell diagnosis device, 4: measuring electrode, 5: culture solution, 6: depression, 7: through hole, 12: Si layer, 13: SiO₂ layer, 14: support substrate, and 19: sample.

FIG. 3 is a schematic diagram showing a cell diagnosis device according to still another embodiment of the present invention. Reference numerals in FIG. 3 indicate the following members. 5: culture solution, 13: SiO₂ layer, 31: spacing section, 35: suctioning line attachment, and 37: cell suctioning system line.

FIG. 4 is a schematic diagram showing a structure of a cell diagnosis apparatus according to an embodiment of the present invention. Reference numerals in FIG. 4 indicate the following members. 101: signal source, 102: unit standard deviation calculating section, 103: normal distribution approximating section, 104: stimulus generating section, 105: average calculating section, 106: average and half-width calculating section, 107: signal adding section, 108: characteristic calculating section, 109: characteristic categorizing section, and 110: data displaying section.

FIG. 5 is a schematic diagram showing a structure of a cell diagnosis apparatus according to another embodiment of the present invention. Reference numerals in FIG. 5 indicate the same members as those indicated with the reference numerals in FIG. 4.

FIG. 6 is a schematic diagram showing a configuration of a cell diagnosis apparatus according to still another embodiment of the present invention. Reference numerals in FIG. 6 indicate the following members. 101: a signal source, 102: unit standard deviation calculating section, 103: normal distribution approximation section, 106: average and half-width calculating section, 109: characteristic categorizing section, 110: data displaying section, and 111: sample number categorizing section.

FIG. 7 is a schematic diagram showing a configuration of a cell diagnosis apparatus according to still another embodiment of the present invention. Reference numerals in FIG. 7 indicate the following members. 101: signal source, 102: unit standard deviation calculating section, 103: normal distribution approximation section, 104: stimulus generating section, 106: average and half-width calculating section, 109: characteristic categorizing section, and 110: data displaying section.

FIG. 8 is a diagram showing Carbachol concentration dependent reactions of normal cells and cells including cancer cells prepared from the fundus of rat stomach, which were measured using a cell diagnosis apparatus according to an embodiment of the present invention.

FIG. 9 is a diagram showing results of measurement before and after Carbachol application of normal cells and cells including cancer cells prepared from the fundus of rat stomach, which were measured using a cell diagnosis apparatus according to another embodiment of the present invention.

FIG. 10 is a diagram showing reactions to 200 Hz pulsed voltage of cells prepared from the normal fundus of rat stomach and the cancerous fundus of rat stomach, which were measured using a cell diagnosis apparatus according to an embodiment of the present invention.

FIG. 11 is a diagram showing reactions to 200 Hz pulsed voltage of normal cell derived membrane fractions and cancer cell derived membrane fractions prepared from the fundus of rat stomach, which were measured using a cell diagnosis apparatus according to an embodiment of the present invention.

FIG. 12 is a schematic diagram showing a cell diagnosis analyzing chip according to an embodiment of the present invention. In this cell diagnosis analyzing chip, a. tissue disruption section, b. cell culturing section, c. sensor section, d. stimulus applying section, e. signal amplifying section, f. data processing section, g. data displaying section, and h. control panel section are provided on a substrate of the cell diagnosis device, whereby a state of a sample can be displayed. Reference numerals in FIG. 12 indicate the following members. 201: sample inlet, 202: cell disruption section, 203: cell culturing section and sensor section, 204: stimulus applying action, 205: signal amplifying section, 206: signal processing section, 207: data displaying section, and 208: control panel section.

### BEST MODE FOR CARRYING OUT THE INVENTION

A method of the present invention for diagnosing a cell, comprises the steps of: providing a reaction system for measuring a physicochemical characteristic of the cell; placing the cell or a cell membrane fraction thereof as a sample in the reaction system; stimulating the sample; obtaining an index of the physicochemical characteristic of the sample, and diagnosing or characterizing a state of cell with reference to the index.

The cell placed in the reaction system may be in the form of a tissue sample, and isolated, free cell, or a fixed cell.

As the stimulus, physical stimuli (e.g., voltage pulse, current pulse, electromagnetic wave, and laser light), chemical stimuli (e.g., contact with a drug), and the like can be used.

A device of the present invention is used in the reaction system in the cell diagnosis method. The device comprises a substrate on which a sample, i.e., a cell or a cell membrane fraction, is placed, and a reference electrode and a measuring electrode, and is characterized in that the placement environment or characteristic of the reference electrode is different from the placement environment or characteristic of the measuring electrode.

The substrate comprises a through hole on which the sample is placed, and the reference electrode and the measuring electrode are provided in the vicinity of the through hole, and the through hole being interposed between the reference electrode and the measuring electrode.

As a material for the substrate, any of semiconductors, insulators, inorganic materials, and organic materials. Examples of the material for the substrate silicon, silicon oxide, silicon-on-insulator (hereinafter referred to as SOI), plastics, rubbers, and polymer films. Among them, SOI and polymer films are preferably used, and more preferably porous polymer films. The thickness of the substrate is preferably 1 to 1000 μm, and more preferably 10 to 100 μm. The diameter of the through hole is preferably 1 to 100 μm, and more preferably 5 to 10 μm.

An example of the above-described placement environment or characteristic is the volume of the regions in which the reference electrode or the measuring electrode is placed. The volume of the region in which the measuring electrode is placed is preferably smaller than the volume of the region in which the reference electrode is placed. In this case, preferably, the volume of the region in which the reference electrode is placed is at least 5 times the volume of the region in which the measuring electrode is placed, and more preferably at least 10 times.

The device typically comprises a cell culturing section placed on the substrate, and a cell suctioning section placed under the substrate. The cell culturing section may be formed of a partitioning member and the substrate. In this case, the shape of the partitioning member is preferably, but not particularly limited, a cylinder. Preferable materials for the partitioning member are silicon, silicon oxide, a plastic, a rubber, or the like. As used herein, the term “sensor section” particularly refers to the device excluding the cell culturing section and the cell suctioning section.

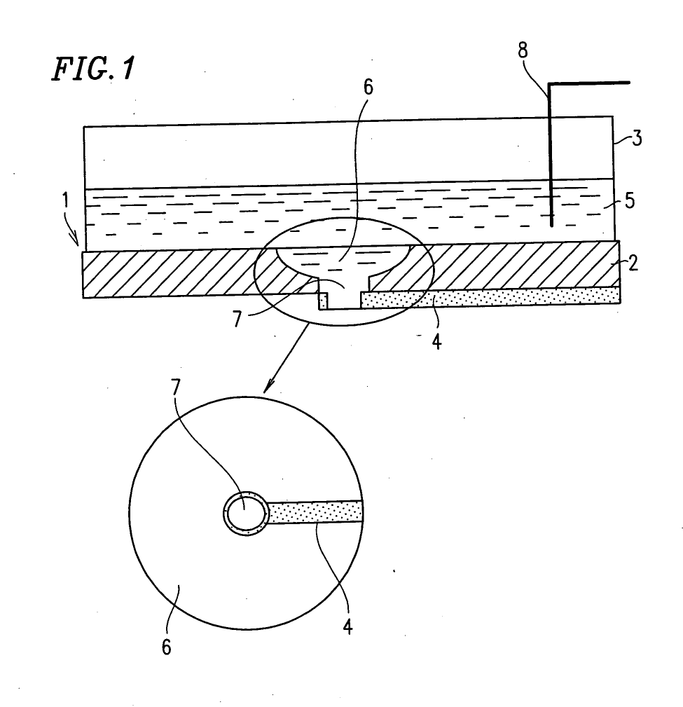

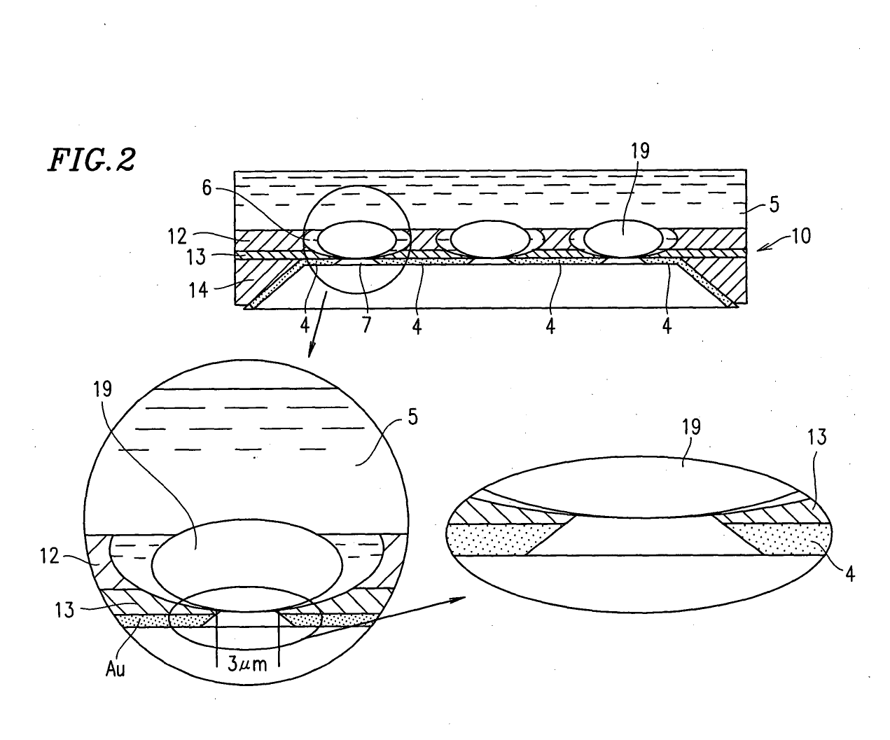

The present invention will be described with reference to FIGS. 1 and 2. FIG. 1 is a cross-sectional view schematically showing an exemplary device used as a reaction system employed in a method of the present invention.

A cell diagnosis device 1 comprises an SOI substrate 2. A well 3 (cell culturing section) for accommodating a sample (a cell or a cell membrane fraction) is provided on an upper surface of the SOI substrate 2. A measuring electrode 4 made of, representatively, gold, for detecting a signal is provided on a lower surface of the SOI substrate 2. About 50 μl of culture solution 5 acting as an electrolyte is present in the well 3. About 1 μl or less of culture solution is present in a through hole 7 provided in the SOI substrate 2.

The well 3 is formed by combination of a partitioning member (not shown) for holding culture solution and the SOI substrate 2. A depression 6, which is provided in the SOI substrate within the well 3, is formed in a shape optimal to hold a cell or a cell membrane fraction. Representatively, the diameter of the opening of the depression 6 is about 10 μm and in the shape of a semi-ellipse. A reference electrode 8 (representatively, Ag-AgC1) is disposed within the well 3, where the reference electrode 8 is immersed in a culture solution 5.

The well 3 accommodating the reference electrode 8, the culture solution 5 immersing the reference electrode 8, and the depression 6 are herein collectively referred to as reference electrode placement environment, and the through hole 7 and the culture solution contained therein are herein collectively referred to as measuring electrode placement environment.

The size of the depression 6 may vary depending on the sample, and is not particularly limited if the sample is held on the depression 6. Representatively, the opening diameter of the depression 6 is in the range of 10 to 500 μm, and the depth thereof is in the range of 1 to 500 μm. Typically, the opening diameter of the depression 6 is in the range of 2 to 100 μm, and the depth thereof is in the range of 2 to 100 μm. For example, a preferable depression has an opening diameter of 20 μm and a depth of 10 μm, and another preferable depression has an opening diameter of 20 μm and a depth of 20 μm. Also, the size of the through hole is not particularly limited as long as sample does not pass through the through hole and the sample is held on the depression. Representatively, the through hole has a diameter in the range of 1 to 100 μm and a depth of 10 nm to 100 μm. A preferable through hole has a diameter of 5 μm and a depth of 1.5 μm, for example.

An enlarged, plan view of the depression 6 is shown in a lower portion of FIG. 1.

FIG. 2 is a schematic diagram showing another exemplary device used in a reaction system in a method of the present invention. A device 10 shown in FIG. 2 is different from the device 1 shown in FIG. 1 in that a plurality of depressions 6 and through holes 7 are provided. As shown in FIG. 2, samples 19 (ellipses in the figure) are held in the respective depression 6. Substrates 12 to 14 are made of SOI a SiO₂ layer 13 is provided under a Si layer 12, and a support substrate 14 is provided under the SiO₂ layer 13. A measuring electrode 4 is provided on a rear surface of the substrates 12 to 14, where the measuring electrode 4 runs along portions of surfaces of the support substrate 14 and the SiO₂ layer 13, while the measuring electrode 4 contacts the sample 19, or is located in the vicinity of the sample 19, preferably within a distance of 10 μm from the sample 19.

In the device used in the reaction system in the method of the present invention, a very small amount of electrolyte is present in the vicinity of the measuring electrode 4. Specifically, the mount of electrolyte present in the vicinity of a surface (i.e., rear surface) of the substrate opposite to another surface of the substrate on which a sample is placed, is no more than about 1 to about 10 μl including electrolyte filling the through hole.

As shown in FIGS. 1 and 2, a depression 6 and a through hole 7 may be provided for each measuring electrode, or alternatively, a plurality of depressions 6 and through holes 7 may be provided for each measuring electrode.

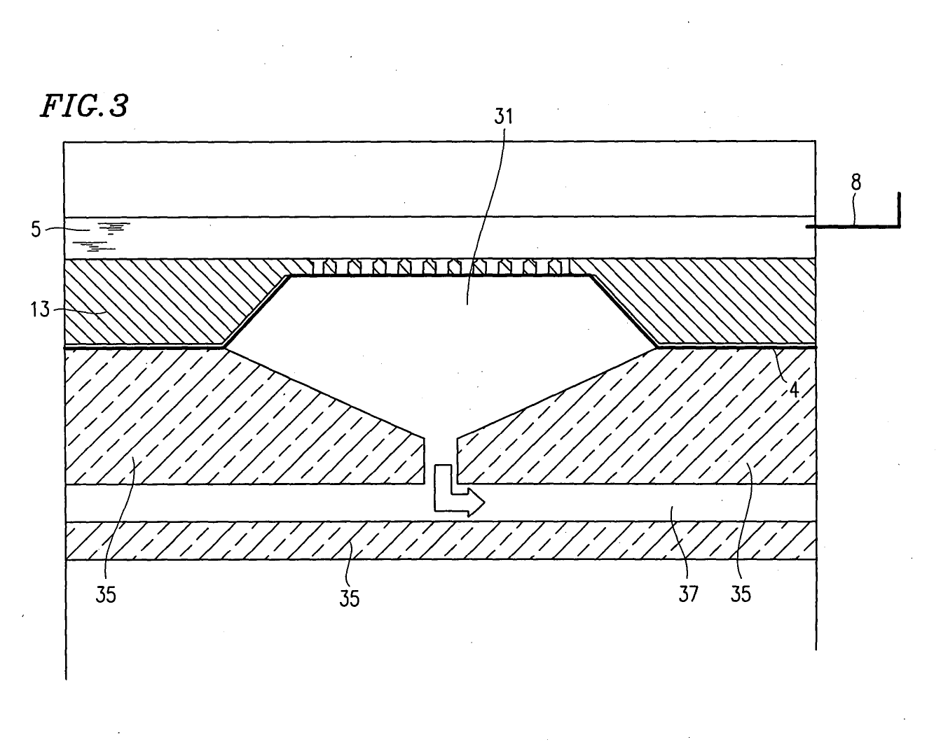

FIG. 3 is a schematic diagram showing a cell suctioning section which is optionally used when the device 10 shown in FIG. 2 is used to measure a physicochemical characteristic of sample, i.e., a cell or a cell membrane fraction. FIG. 3 shows a cell suctioning section comprising a suctioning line attachment 35 and a cell suctioning system line 37 which are provided on the rear surface of the SiO₂ layer 13 constituting the substrate. The suctioning line attachment 35 is made of a material, such as acrylic resin, PMDS, silicone rubber, or the like, forming spacing sections 31 which are in communication with the cell suctioning system line 37 and which correspond to respective through hole 7 provided in the substrate. The suctioning line attachment 35 can be adhered to an integrated with the substrate. As shown in FIG. 3, when measuring a physicochemical characteristic of a cell or a cell membrane fraction, the sample, i.e., the cell or the cell membrane fraction, is tightly attached to the substrate preferably by applying suctioning pressure from the cell suctioning system line 37. In this case, the cell suctioning section does not have to be completely filed with electrolyte as long as a physicochemical characteristic of the sample, i.e., the cell or the cell membrane fraction, can be detected with the measuring electrode. Note that in FIG. 3, the sample, i.e., the cell or the cell membrane fraction, are not shown, and the structures of the depression and the through hole are simplified.

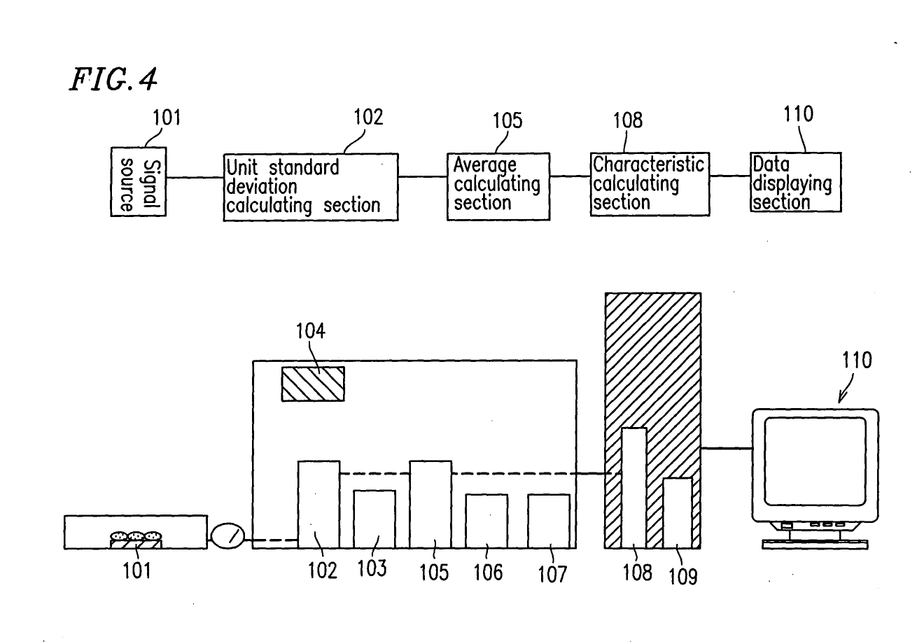

FIG. 4 is a conceptual diagram showing a structure of a cell diagnosis apparatus comprising a device of the present invention, for determining the state of a subject cell. The apparatus comprises a measure section (signal source) 101 comprising the device, a unit standard deviation calculating section 102 which samples a signal from the measuring section 101, and calculates standard deviations, an average calculating section 105 which calculates an average of the resultant standard deviations, and a characteristic calculating section 108 which calculates a physicochemical characteristic of the sample, i.e., the cell or the cell membrane fraction, from the average standard deviation output from the average calculating section 105, and a data displaying section 110 which displays the resultant physicochemical characteristic. Connections between each section are indicated with dashed or solid lines in FIG. 4. Note that representatively, the unit standard deviation calculating section 102, the average calculating section 105, and the characteristic calculating section 108 are programs for executing the above-described calculations, which are stored in a hard disk incorporated into a computer. The data displaying section 110 is a CRT.

Note that in FIG. 4, reference numerals 103, 104, 106, 107, and 109 indicate a normal distribution approximation section, a stimulus generating section, an average/half width calculating section, a signal adding section, and an activity categorizing section, respectively, which are described below.

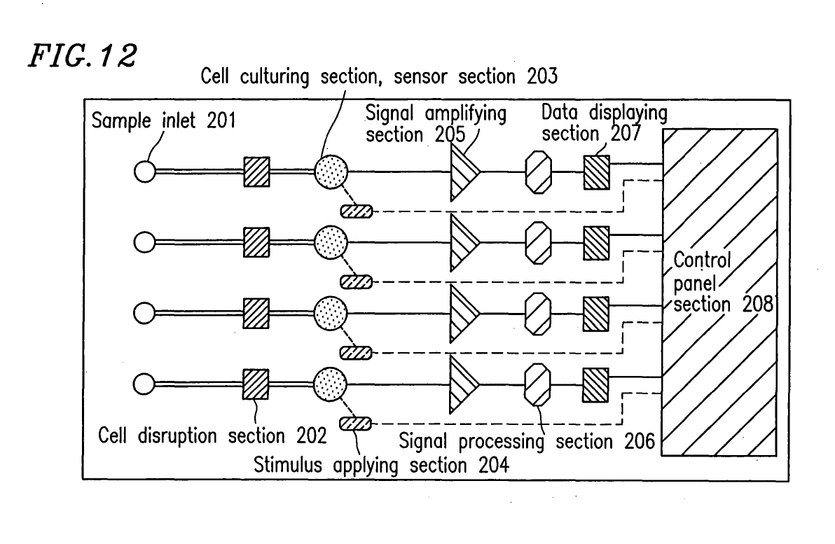

FIG. 12 schematically shows a cell diagnosis analyzing chip according to an embodiment of the present invention. This chip comprises a substrate on which a cell or its cell membrane fraction is provided as sample, a reference electrode, and a measuring electrode. On this substrate, provided are a sample inlet 201, a cell disruption section 202, a cell culturing section and a sensor section (which are integrated together into a member indicated by reference numeral 203 in FIG. 12), a stimulus applying section 204, a signal amplifying section 205, a signal processing section 206, a data displaying section 207, and a control panel section 208. With this configuration, a series of steps for diagnosing cells are conducted and the results can be displayed.

A sampled injected through the sample inlet 201 is introduced via a flow path, which is in fluid communication with the cell disruption section 202, to the cell disruption section 202. The cell disruption section 202 representatively comprises a microfilter having a diameter of 20 μm (e.g., available from Millipore), thereby recovering a sample to be tested, such as the cell or its cell membrane fraction placed in the cell culturing section. Thereafter, the recovered sample is introduced to the cell culturing section via the flow path that is in fluid communication with the cell culturing section. The cell culturing section is placed on a sensor section (i.e., a device as described herein) comprising a reference electrode and a measuring electrode. The introduced sample is stimulated in the sensor section by the stimulus applying section 204 under the control of the control panel section 208, thereby generating a signal which is an index of the physicochemical characteristic of the sample.

Typically, the cell culturing section comprises a temperature control means for maintaining the sample in an activated state, and a humidity control means. As the temperature control means, a Peltier device, an IH heater, or the like can be used. A generated signal is amplified by the signal amplifying section 205 connected to the sensor section, is then processed by the signal processing section 206, and is then transferred to the data displaying section 207, in which the processing results are displayed. Note that as elements constituting the above-described members and the connection between each member, elements known to those skilled in the art may be used, and they are not particularly explained here. Further, according to the example shown in FIG. 12, the cell diagnosis analyzing chip comprises four lines each comprising the above-described members for performing the above-described series of steps. The number of lines is not so limited.

A method, a device, and a cell diagnosis apparatus of the present invention provide a novel method for diagnosing a cell by providing a simple device and apparatus for extracting a signal which cannot be detected by conventional extracellular methods.

A cell diagnosis method of the present invention does not employ an antibody, and therefore, does not require a staining step necessary for methods using antibodies. Errors in diagnosis due to antibody titer do not occur. The cell diagnosis method of the present invention measures a physicochemical characteristic of a cell or its cell membrane fraction so as to detect a mutation in the subject cell to be diagnosed, whereby a large amount of samples can be processed at a high rate and the process can be automated.

A cell diagnosis method of the present invention representatively processes a digital signal (time-series signal values) sampled at a predetermined sampling rate in the step of obtaining indexes of the physicochemical characteristic of a cell or its cell membrane fraction, thereby making it possible to extract, measure, and categorize the physicochemical characteristic of the cell or its cell membrane fraction as a signal significantly distinct from a noise signal.

A device and a cell diagnosis apparatus of the present invention do not require a dedicated control apparatus, and makes it possible to measure the extracellular potential of a sample (i.e., a cell or a cell membrane fraction) in a short time by merely placing the sample on a substrate without forming high-resistance seal (gigaseal) between the sample and the substrate.

A device of the present invention makes it possible to measure a physicochemical characteristic of a cell or a cell membrane fraction by the changing of the placement environment of a measuring electrode or a reference electrode without forming a high-resistance seal (gigaseal) between the sample and a substrate.

### EXAMPLES

Hereinafter, specific examples of the present invention will be described with reference to the drawings.

The present invention will be described by way of examples. The present invention is not so limited.

### Example 1

A cell diagnosis apparatus comprising the device shown in FIG. 3 as a measuring section (signal source) 101, which has a configuration shown in FIG. 4, was used. Normal cells and cancer cells were prepared as samples from the fundus of rat stomach. The apparatus was used to measure the action of a chemical substance, Carbachol, on these samples.

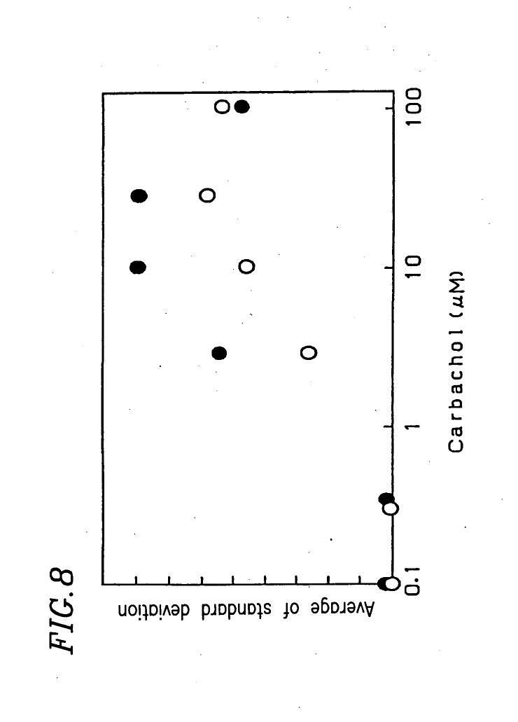

Carbachol is a chemical substance known as an analog of the neurotransmitter Acetylcholine. Carbachol (manufactured by Sigma) was dissolved in Krebs Ringer solution to concentrations of 0, 0.1, 0.3, 1, 3 10, 30 and 100 μM. The solutions having these concentrations were used to measure an electrical signal when each solution was applied to the normal cells and the cancer cells derived from the fundus of stomach. For each Carbachol concentration, time-series signal values for 10 seconds were obtained from the measuring section (signal source) 101 comprising the cell diagnosis device shown in FIG. 3, and was sampled at intervals of 100 msec to obtain time-series data, and the standard deviation of the sampling data was calculated. The average of the standard deviation was plotted in FIG. 8. In FIG. 8, black circles indicate results obtained from cells including cancer cells and white circles indicate results obtain from normal cells.

As shown in FIG. 8, it was confirmed that for either normal or cancer cells, the higher the Carbachol concentration, the higher the average of the standard deviation at 100 msec intervals. This shows that ion channels of the cell prepared from the fundus of rat stomach were activated depending on the Carbachol concentration.

As shown in FIG. 8, the cancer cell had a larger standard deviation of the obtained electrical signal than that of the normal cell, and exhibited a Carbachol concentration dependent pattern different from that of the normal cell, where Carbachol concentration was 1 to 30 μM. Therefore, it was found that a method and a cell diagnosis apparatus of the present invention can be used to measure a physicochemical characteristic of a cell and determine the presence or absence of a cancel cell.

### Example 2

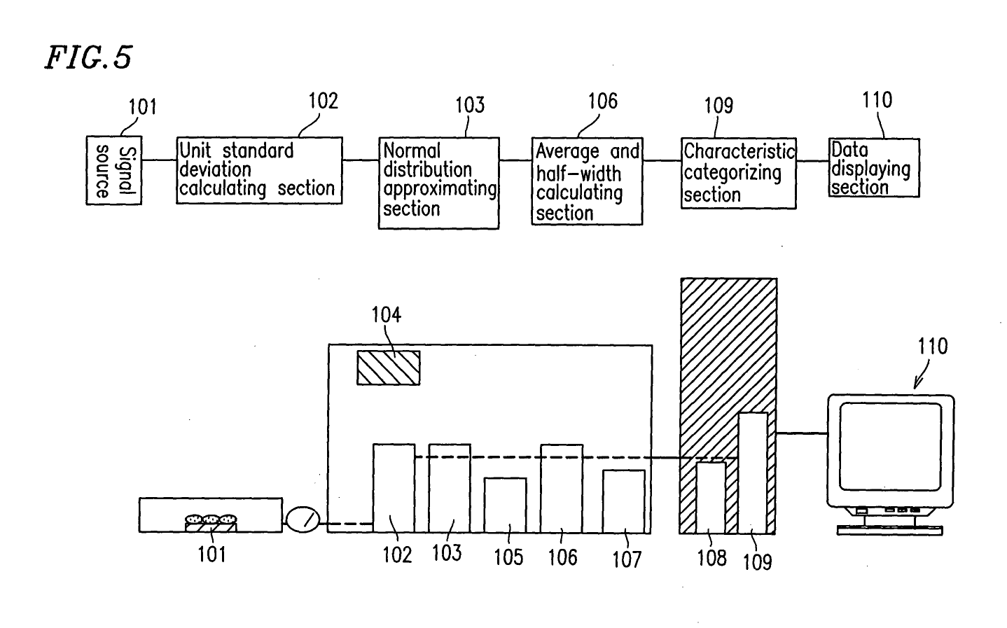

FIG. 5 is a conceptual diagram showing a configuration of a cell diagnosis apparatus for determining the state of a subject cell according to the present invention. The apparatus is the same as the apparatus of FIG. 4, except that a normal distribution approximation section 103, an average/half-width calculating section 106 and an activity categorizing section 109 are employed in place of the average calculating section 105 and the characteristic calculating section 108. Communications between each section are indicated by dashed lines or solid lines in FIG. 5.

The normal distribution approximation section 103 divides a plurality of standard deviation values obtained by the unit standard deviation calculating section 102 into a plurality of classes having a predetermined width of standard deviation as a unit, plots the standard deviation values where the X axis represents the class and the Y axis represents the number of standard deviation values belonging to the class, and the approximates the obtained graph to a normal distribution. The average/half-width calculating section 106 calculates the average and half-width of the resultant normal distribution. The activity categorizing section 109 categorizes a physicochemical characteristic based on the obtained average and half-width. Note that similar to the apparatus of FIG. 4, the unit standard deviation calculating section 102, the normal distribution approximation section 103, the average/half-width calculating section 106 and the activity categorizing section 109 may be representatively software programs which are recorded in a hard disk of a computer. The data displaying section 110 may be a CRT.

The apparatus having the configuration shown in FIG. 5, which comprises the device shown in FIG. 3 as a measuring section (signal source), was used to measure the action of Carbachol on samples, where the samples were normal cells and cells including cancer cells prepared from the fundus of rat stomach, as in Example 1.

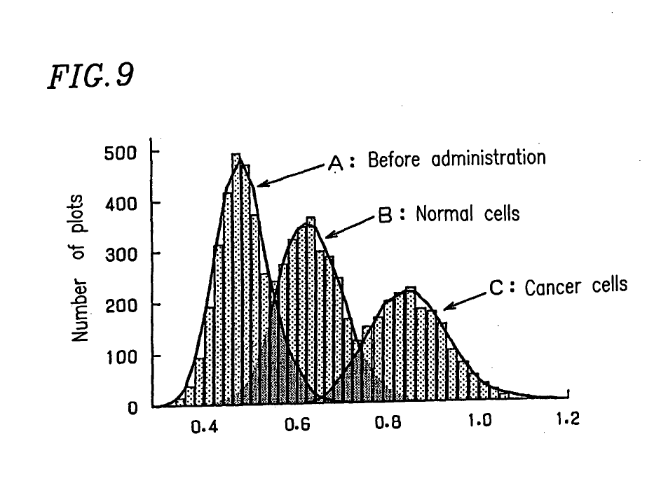

Before and after 50 μM-concentration Carbachol was applied to the normal cells and the cancer cells prepared from the fundus of rat stomach, signals were obtained from the measuring section (signal source) 101, and the standard deviation of the signals was calculated in a manner similar to that of Example 1. The normal distribution approximation section 103 plotted the standard deviations into a graph which is shown in FIG. 9.

FIG. 9(A) shows a histogram of standard deviation values calculated from time-series data of an electrical signal every 5 msec for 10 seconds before applying Carbachol to the normal cells and the cancer cells. FIG. 9(B) shows a histogram of standard deviation values calculated from time-series data of an electrical signal every 5 msec for 10 seconds, after applying Carbachol to the normal cells. FIG. 9(C) shows a histogram of standard deviation values calculated from time-series data of an electrical signal for 10 seconds every 5 msec after applying Carbachol to the cancer cells. As shown in FIG. 9, it was confirmed that the average of the standard deviation every 5 msec was increased after applying Carbachol for the normal cells and the cancer cells. Further, it was confirmed that the cancer cell had larger average and half-width values than those of the normal cell. Therefore, it was found that a method and a cell diagnosis apparatus of the present invention can be used to measure a physicochemical characteristic of a cell and determine the presence or absence of a cancer cell.

### Example 3

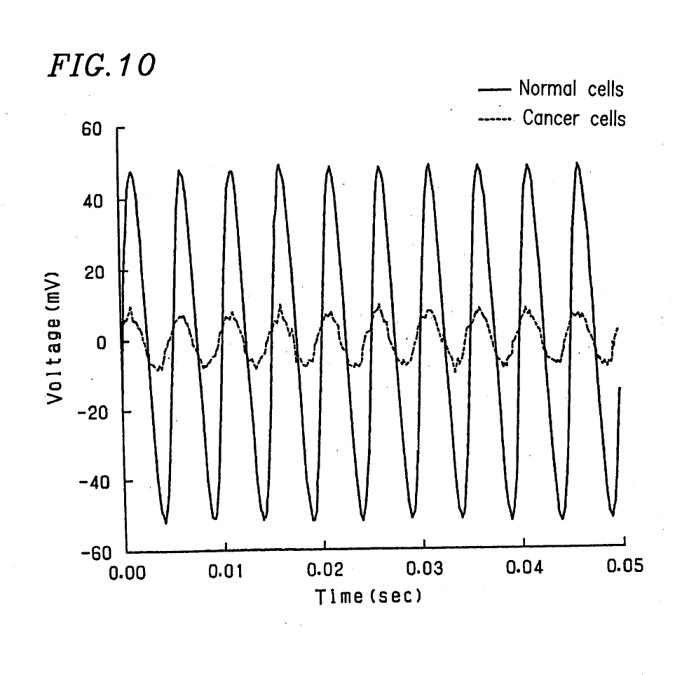

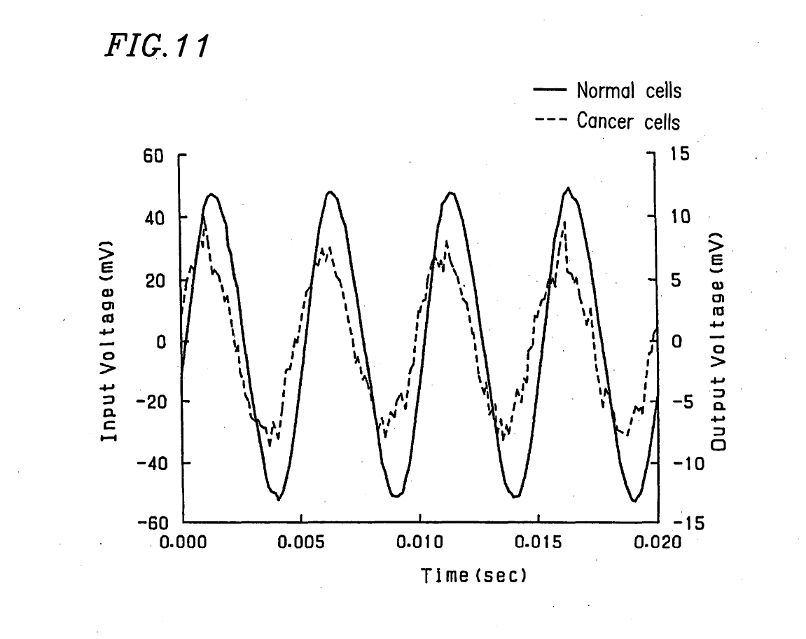

As in Example 1, cells were prepared as samples from the normal fundus of rat stomach and the cancerous fundus of rat stomach, the samples were stimulated by apply 200 Hz pulse voltage, and a voltage signal generated from the samples were measured. Results are shown in FIG. 10. Further, normal cell-derived membrane fractions and cancer cell-derived membrane fractions were used as samples and were subjected to a similar experiment. The results were shown in FIG. 11. In each figure, the solid line indicates the measurement results from the normal cell samples, and the dashed line indicates the measurement results from the cancer cell samples.

As shown in FIGS. 10 and 11, either when cells were used as samples or when cell membrane fractions were used as samples, it was found that the amplitude of the voltage signal was attenuated in the cancer cell, as compared to the normal cell. Therefore, it was found that a method and a cell diagnosis apparatus of the present invention can be used to measure a physicochemical characteristic of a cell and determine the presence or absence of a cancer cell.

In this example, a unit standard deviation calculating section as shown in FIG. 4 was not used, and only the attenuation of the voltage signal was used for comparison. However, various frequencies can be applied and then impedance changes or a volume component can be analyzed, resulting in more detailed analysis.

As described above, it was revealed that by employing a method and a cell diagnosis apparatus of the present invention in place of conventional immunostaining, a physicochemical characteristic of a cell or a cell membrane fraction can be simply measured and extracted to determine the state of the cell. In the above-described examples, a physicochemical characteristic of a plurality of cells or cell membrane fraction prepared from the cells was measured in a reaction system, however, similar results can be obtained when a single cell is measured.

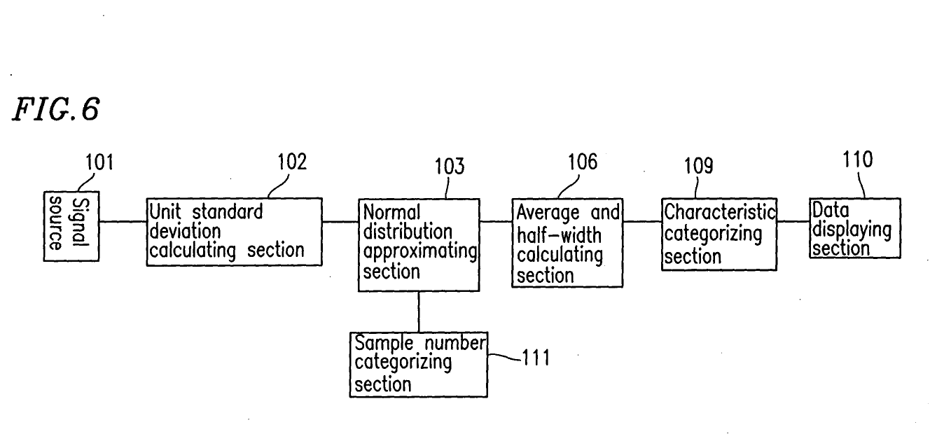

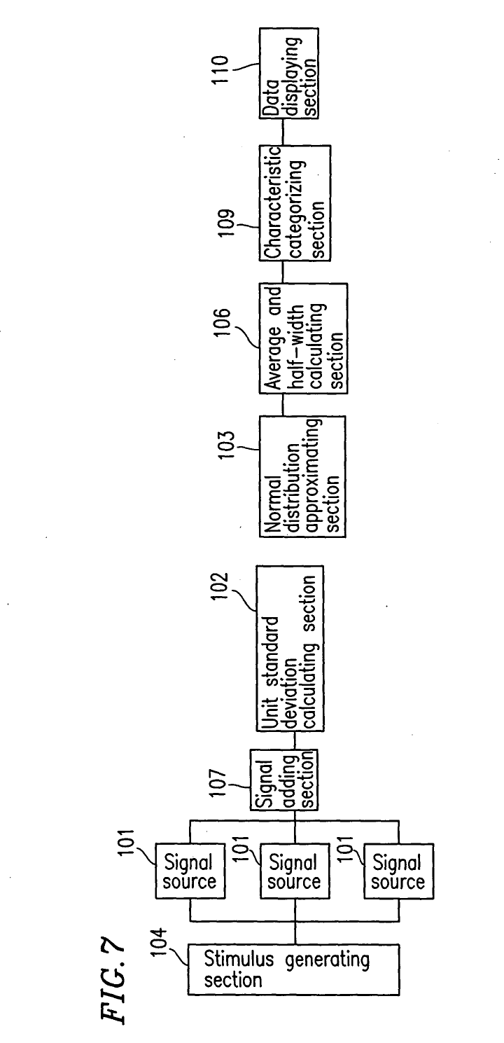

Note that although the apparatus schematically shown in FIG. 4 or 5 was used in the above-described examples, an apparatus having the configuration schematically shown in FIG. 6 or 7 may be used instead.

An apparatus shown in FIG. 6 comprises a sample number categorizing section indicated by reference numeral 111 in addition to the apparatus shown in FIG. 5.

An apparatus shown in FIG. 7 is the same as the apparatus of FIG. 5, except that the apparatus of FIG. 7 comprises a plurality of signal sources 101, a signal generating section 104 for stimulating the signal sources 101, and a signal adding section 107 for adding signals from the signal sources 101.

### Industrial Applicability

A cell diagnosis method, and a device and an apparatus using the same, in which by measuring changes in a physicochemical characteristic due to a mutation in a cell, a large amount of samples can be easily processed in a short time, are provided.

1. A method for diagnosing a state and a characteristic of a cell, comprising the steps of:
providing a reaction system for measuring a physicochemical characteristic of the cell;
placing a sample including a cell membrane fraction of the cell in the reaction system;
applying a stimulus to the sample;
obtaining an index of the physicochemical characteristic of the sample; and
diagnosing the state of the cell with reference to the index.

2. A method according to claim 1, wherein the diagnosing step comprises comparing the index with an index of the physicochemical characteristic of a control sample.

3. A method according to claim 1, wherein the stimulus is selected from chemical substances, proteins, amino acids, voltage pulses, current pulses, electromagnetic waves, and laser light.

4. A device for use in the reaction system of claim 1, comprising:
a substrate for placing the sample thereon; a reference electrode; and a measuring electrode, wherein
the placement environment or characteristic of the reference electrode is different from the placement environment or characteristic of the measuring electrode.

5. A device according to claim 4, wherein the substrate comprises a through hole for placing the sample thereon; and the sample is placed between the reference electrode and the measuring electrode and in the vicinity of the through hole.

6. A device according to claim 4, wherein the placement environment or characteristic of the reference electrode and the placement environment or characteristic of the measuring electrode are the volumes of regions in which the reference electrode and the measuring electrode are disposed, respectively; and the volume of the region in which the measuring electrode is disposed is smaller than the volume of the region in which the reference electrode is disposed.

7. A method according to claim 1, wherein the step of providing the reaction system comprises supplying a sufficient amount of an electrolyte to the device of claim 5 that the reference and measuring electrodes are immersed in the electrolyte, and
the placing step comprises positioning the sample on a through hole of the device,
wherein the amount of the electrolyte immersing the reference electrode is greater than the amount of electrolyte immersing the measuring electrode.

8. A device according to claim 5, further comprising a cell culturing section disposed on the substrate, and a cell suctioning section disposed under the substrate, wherein the cell culturing section is formed by a partitioning member and the substrate.

9. A device according to claim 8, wherein the reference electrode is disposed on an inner wall of the partitioning member.

10. A method according to claim 1, wherein the index obtaining step comprises:
(a) recording a physicochemical signal emitted by the sample as time-series signal values;
(b) sampling the recorded time-series signal values to obtain a plurality of groups of extracted data consisting of a plurality of values, and calculating a standard deviation of each group of data;
(c) calculating an average value of the standard deviations; and
(d) referring to the average as the index.

11. A method according to claim 1, wherein the index obtaining step comprises:
(a) recording a physicochemical signal emitted by the sample as time-series signal values;
(b) sampling the recorded time-series signal values to obtain a plurality of groups of extracted data consisting of a plurality of values, and calculating a standard deviation of each group of data;
(c) dividing the standard deviations into a plurality of classes having a predetermined size of standard deviation as a unit, and obtaining a distribution indicating the physicochemical characteristic of the sample;
(d) approximating the distribution to a normal distribution; and
(e) calculating an average and a half-width of the resultant normal distribution, and referring to the average and the half-width as the indexes.

12. A method according to claim 11, wherein the steps (b) to (e) are repeated a plurality of times, the number of the timer-series signal values to be sampled is changed in each repetition to obtain a plurality of normal distributions, and the index is selected from averages and half-widths of the normal distributions.

13. A method according to claim 11, wherein there are a plurality of reaction systems for measuring a physicochemical characteristic of the cell, and before the step (b), the method further comprises adding up the time-series signal values emitted by a plurality of samples placed in the reaction systems.

14. A method according to claim 13, wherein before the step (a), the method further comprises simultaneously stimulating the samples placed in the reaction systems.

15. A method according to claim 1, wherein the index obtaining step comprises:
(a) recording a physicochemical signal emitted by the sample as time-series signal values;
(b) sampling the time-series signal values to obtain a plurality of groups of extracted data consisting of a plurality of values, and calculating a standard deviation of each group of extracted data;
(c) dividing the standard deviations into a plurality of classes having a predetermined size of standard deviation as a unit, and obtaining a distribution indicating the physicochemical characteristic of the sample;
(d) approximating the distribution by curvilinear approximating analysis selected from the group consisting of exponential decreasing analysis, exponential increasing analysis, Gaussian distribution, Lorentz distribution, o′ analysis, multiple peak analysis, and nonlinear analysis; and
(e) obtaining an index of the physicochemical characteristic of the sample based on gradients before and after a peak on the approximated curve obtained by the step (d).

16. A method according to claim 10, wherein the step (b) is carried out a plurality of times from initial data a, which is one of the time-series signal values, in a time-series manner and a plurality of times from data b recorded at a predetermined time after the initial data a in a time-series manner.

17. A method according to claim 1, wherein the index obtaining step comprises:
(a) recording a physicochemical signal emitted by the sample as time-series signal values;
(b) sampling the time-series signal values to obtain a plurality of groups of extracted data consisting of a plurality of values, and calculating a standard deviation of each extracted data;
(c) sampling the resultant standard deviations to obtain a plurality of values, and calculating an average of each of the plurality of groups of extracted standard deviations; and
(d) obtaining an index of the physicochemical characteristic of the sample based on a time of occurrence of the time-series signal value when the average reaches a predetermine threshold.

18. A method according to claim 11, wherein the step (a) is performed in the presence of a standard chemical substance having a known action on the sample and in the presence of a chemical substance to be tested, and the steps (b) to (e) are repeated by changing the concentrations of the standard chemical substance and the chemical substance to be tested, and
the diagnosing step further comprises comparing an index obtained in the presence of the standard chemical substance with an index obtained in the presence of the chemical substance to be tested.

19. A cell diagnosis apparatus, comprising a reaction system comprising the cell diagnosis device of claim 8, and means for detecting the physicochemical characteristic of the sample.

20. A cell diagnosis analyzing chip, comprising a substrate for placing a sample including a cell membrane fraction of a cell thereon, a reference electrode, and a measuring electrode,
wherein the substrate has a. tissue disruption section, b. cell culturing section, c. sensor section, d. stimulus applying section, e. signal amplifying section, f. signal processing section, g. data displaying section, and h. control panel section, thereby displaying the state of the sample.

21. A cell diagnosis chip according to claim 20, wherein the a. tissue disruption section comprises a microfilter having a diameter of 1 to 100 μm.

22. A cell diagnosis chip according to claim 20, wherein the b. cell culturing section has temperature control means.

23. A cell diagnosis chip according to claim 22, wherein the b. cell culturing section further has the humidity control means.

24. A cell diagnosis chip according to claim 22, wherein the temperature control means has a Peltier device or an IH heater.
